(19) United States
(12) Patent Application Publication
(10) Pub. No.: US 2026/0007889 A1
(43) Pub. Date: Jan. 8, 2026

(54) TREATING RESPIRATORY CONDITIONS USING SPINAL CORD STIMULATION

(71) Applicant: Spiro Medical, Inc., Belleair Bluffs, FL (US)

(72) Inventor: Stephen T. Pyles, Ocala, FL (US)

(73) Assignee: Spiro Medical, Inc., Belleair Bluffs, FL (US)

(21) Appl. No.: 18/761,976

(22) Filed: Jul. 2, 2024

(51) Int. Cl. A61N 1/36; A61N 1/05

(52) U.S. Cl. CPC A61N 1/36062; A61N 1/0551; A61N 1/3606; A61N 1/36157; A61N 1/36171; A61N 1/36175

(57) ABSTRACT

A device for treating respiratory conditions is disclosed. The device generates electrical pulses directed to a region of the spinal cord column. The electrical pulses provide a neuromodulation that prevents bronchoconstriction to mitigate symptoms associated with chronic respiratory conditions such as asthma and chronic obstructive pulmonary disease (COPD).

### BACKGROUND

Asthma is a chronic condition characterized by constriction of airways or bronchioles, often coinciding with excess mucus production. This phenomenon often causes difficulty breathing and can be triggered by exercise, use of certain medications, contact with common workplace irritants or allergens, and by cytokines, eosinophils, and immunoglobulin E (IgE).

Symptoms of asthma typically include shortness of breath, wheezing, coughing, and chest tightness. When the muscles around the bronchioles become constricted, a bronchospasm, also called an asthma attack, may onset, during which symptoms rapidly worsen. Patients are known to visit emergency rooms and undergo potential hospitalizations due to sudden and severe asthma attacks, which can lead to respiratory failure, unconsciousness, or even death.

While treatments and medications can be used to manage symptoms, there is no known cure for asthma. Furthermore, the treatments that are available for managing asthma symptoms are often accompanied by inconsistent symptom relief and common side effects.

Traditionally, patients receive short-term and long-term medications, also known as bronchodilators, that require frequent administration and impose a substantial burden on patients' daily lives. Long-term control medications, such as inhaled corticosteroids (ICS) combined with long-acting beta agonists (LABA), are typically used for asthma management, aiming to prevent attacks and manage chronic symptoms. These medications are typically taken twice per day and aim to reduce swelling in the airways and lungs. In cases where asthma symptoms need to be resolved more rapidly, quick-relief medications, like short-acting beta agonists (SABA), can provide relief. These medications often are referred to as “rescue inhalers” and act over the course of 15 and 20 minutes, with relief of symptoms lasting between 4 and 6 hours.

Biologic medications have recently been developed to treat asthma. Biologic medications target underlying biological mechanisms that result in bronchospasm in severe asthma cases. However, not all of these medications work for every patient, which often necessitates a trial-and-error approach in determining which medication will be the most effective for a given case. Additionally, biologic medications may present significant side effects, such as inflammation, tremors, nausea, and allergic reactions such as facial swelling, rashes, and hives.

### SUMMARY

In general terms, the present disclosure relates to treating respiratory conditions by stimulating neural tissue proximate to and/or inside of a targeted area of an epidural space. In one possible configuration, a device is programmed to generate electrical pulses to provide a neuromodulation signal having an amplitude, a frequency, and a pulse width set to prevent onset of an asthma attack. Various aspects are described in this disclosure, which include, but are not limited to, the following aspects.

One aspect relates to a device for treating respiratory conditions comprising: a processing circuitry having non-transitory computer readable storage media storing instructions which, when executed by the processing circuitry, cause the processing circuitry to: generate electrical pulses directed to a region of the spinal cord column, the electrical pulses providing a neuromodulation that prevents bronchoconstriction, the neuromodulation having an amplitude ranging from about 0.5 mAmp to about 2.5 mAmp, a frequency ranging from about 350 Hz to about 500 Hz, and a pulse width ranging from about 240 μs to about 350 μs.

Another aspect relates to a method of treating respiratory conditions comprising: acquiring a pulse generator configured for neuromodulation; and programming the pulse generator to generate electrical pulses directed to a region of the spinal cord, the electrical pulses providing the neuromodulation to prevent bronchoconstriction, the neuromodulation having an amplitude ranging from about 0.5 mAmp to about 2.5 mAmp, a frequency ranging from about 350 Hz to about 500 Hz, and a pulse width ranging from about 240 μs to about 350 μs.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system that can be used to mitigate symptoms caused by chronic respiratory conditions.

FIG. 2 illustrates a spine anatomy in which the system of FIG. 1 is implanted.

FIG. 3 illustrates examples of additional types of stimulators that can be used to mitigate symptoms caused by chronic respiratory conditions.

FIG. 4 schematically illustrates an example of the system of FIG. 1 having a distal end of a lead implanted in proximity to neural tissue in a targeted area of the epidural space, and a proximal end of the lead plugged into a pulse generator.

FIG. 5 schematically illustrates another example of a stimulator of FIG. 3 having a distal end implanted in proximity to neural tissue in the targeted area of the epidural space.

FIG. 6 schematically illustrates another example of a stimulator of FIG. 3 having a distal end implanted in proximity to neural tissue in the targeted area of the epidural space.

FIG. 7 schematically illustrates an example of a method of treating respiratory conditions using the system of FIG. 1 or the stimulators of FIG. 3.

FIG. 8 is a chart showing average monthly asthma and COPD medication dosage over duration of time by subjects of a pilot study who received implantation of the system of FIG. 1 programmed to operate with neuromodulation parameters within value ranges identified as targeting bronchodilation and/or preventing bronchoconstriction.

FIG. 9 illustrates a chart showing average number of medication doses taken per month over time for an asthma-only subject group within the pilot study of FIG. 8 and after implantation of the system of FIG. 1.

FIG. 10 illustrates a chart showing asthma control test (ACT) scores for the subjects of the pilot study of FIG. 8 after implantation of the system of FIG. 1.

FIG. 11 illustrates a chart showing asthma control questionnaire (ACQ) scores for the subjects of the pilot study of FIG. 8 after implantation of the system of FIG. 1.

FIG. 12 illustrates a chart showing asthma quality of life questionnaire (AQLQ) scores for the subjects of the pilot study of FIG. 8 after implantation of the system of FIG. 1.

FIG. 13 schematically illustrates an example of a processing circuitry that can be included in the pulse generator of the system of FIG. 1, the stimulators of FIG. 3, and external controllers in FIGS. 4-6.

### DETAILED DESCRIPTION

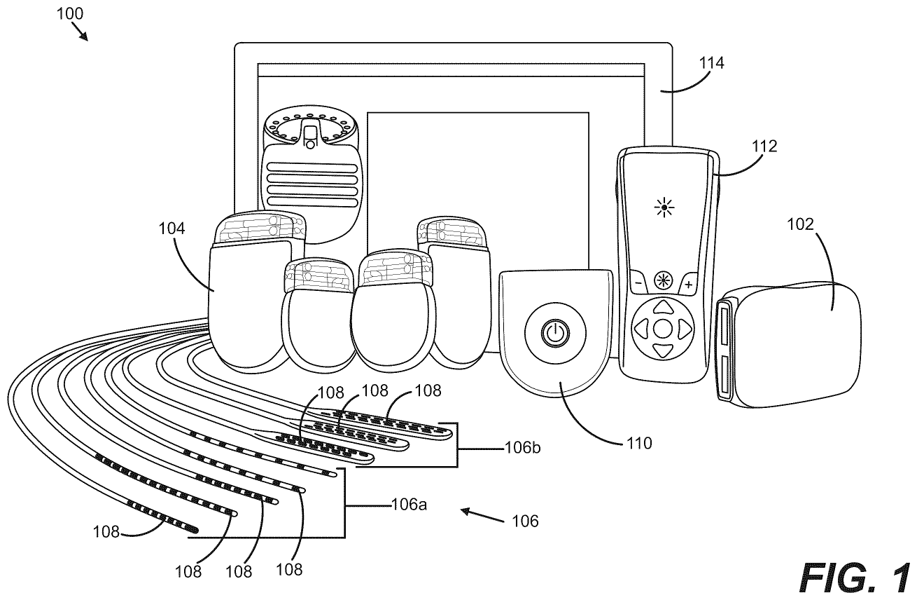

FIG. 1 illustrates an example of a system 100 that is designed to be implanted into a patient to deliver neuromodulation to tissue surrounding the spinal cord to treat and mitigate chronic pain. As will be described, the system 100 can be programmed to additionally mitigate symptoms caused by chronic respiratory conditions such as asthma, chronic obstructive pulmonary disease (COPD), and other chronic pulmonary diseases.

Following a trial stimulation with an external trial stimulator 102, a pulse generator 104 is permanently implanted in the upper buttock or lower flank of a patient, and leads 106 are implanted within a targeted area of an epidural space of the spinal cord column. In this example, the leads 106 are designed to be physically connected to the pulse generator 104 to receive programmed electrical signals that are generated by the pulse generator 104. The leads 106 release the electrical pulses in the targeted area of the epidural space to provide neuromodulation of neural tissues to mitigate symptoms associated with chronic pulmonary diseases.

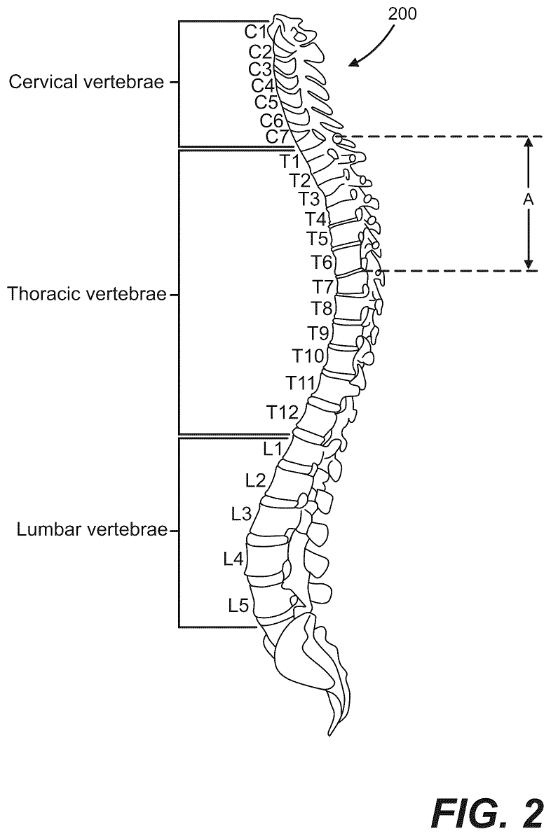

FIG. 2 illustrates a spine anatomy 200 in which the system 100 is implanted. A targeted area A of the epidural space for implanting the leads 106 is shown. The targeted area A of the epidural space for implanting the leads 106 is between the C7 and T6 vertebrae. In some instances, the targeted area A of the epidural space for implanting the leads 106 is between the T1 and T4 vertebrae. The targeted area A of the epidural space allows the neuromodulation provided by the system 100 to stimulate a dorsal spinal cord region causing bronchodilation of the bronchial smooth muscles. The neuromodulation targets directly the sympathetic nervous system and indirectly inhibits the parasympathetic pulmonary axis to treat asthma.

The neuromodulation targets sympathetic ganglia between the C7 and T6 vertebrae. In some instances, the neuromodulation targets sympathetic ganglia between the T1 and T4 vertebrae. In this region, the sympathetic ganglia serve the bronchus and bronchiole causing the neuromodulation to prevent bronchoconstriction, and thereby mitigate symptoms associated with moderate and severe asthma. The neuromodulation provided by the system 100 when programmed in accordance with the neuromodulation parameters described herein can prevent onset of bronchospasms, also called an asthma attacks, without the need for drug intervention.

As shown in FIG. 1, the system 100 can use percutaneous leads 106a that can be permanently implanted inside the targeted area A of the epidural space. Alternatively, the system 100 can use paddle leads 106b that can be permanently implanted inside the targeted area A.

The percutaneous leads 106a and the paddle leads 106b each include electrical contacts 108 that can be cylindrical in the case of the percutaneous leads 106a, or that can be planar in the case of the paddle leads 106b. The electrical contacts 108 release the electrical pulses inside the targeted area A of the epidural space to provide neuromodulation that prevents bronchoconstriction, and thereby mitigates symptoms associated with chronic respiratory conditions such as asthma, COPD, and other pulmonary diseases.

As an illustrative example, the system 100 can utilize percutaneous leads having 16 of the electrical contacts 108 at the distal ends of the leads. The leads plug into the pulse generator 104 at the proximal ends of the leads. For example, a torque wrench can be used to tighten set screws that lock the leads 106 into the pulse generator 104. Additionally, the system 100 can include lead splitters to connect multiple leads to the pulse generator 104.

The system 100 also includes multiple non-implantable components to aid with device setup prior to implantation, as well as communication between components following implantation. For example, the external trial stimulator 102 is used prior to permanent implantation of the pulse generator 104 to gauge the patient's response to spinal cord stimulation. The external trial stimulator 102 is intended to provide trial stimulation, identical stimulation capabilities as the pulse generator 104, to the implanted leads such that the neuromodulation parameters may be fine-tuned to fit the patient's specific needs.

To facilitate the tuning and adjustment of stimulation parameters, the system 100 includes a clinician programmer 114. In some examples, such as the one shown in FIG. 1, the clinician programmer 114 is a portable tablet computer. The clinician programmer 114 is installed with programming software, and a programming wand will be used to allow the clinician programmer 114 to communicate wirelessly with the external trial stimulator 102 and the pulse generator 104. After implantation of the leads 106, providers will maintain control of a remote control 112. As shown in FIG. 1, the remote control 112 is a handheld, battery-operated unit that uses telemetry to communicate with the stimulators, allowing the provider to control the stimulation therapy (e.g., turn the SCS system on and off). The pulse generator 104 once implanted can remain on at all times to ensure stimulation in constantly occurring to prevent onset of bronchoconstriction. The system 100 can include a charger 110 for transcutaneous charging of an internal power source (e.g., battery) of the pulse generator 104.

The system 100 can include additional accessories such as stylets that can be used to maneuver the leads 106 through the epidural space to the desired implant location; a template that can be used to guide the physician to create the correct sizing of a subcutaneous pocket for implanting the pulse generator 104; an insertion needle that can be used during implant procedures to introduce the percutaneous leads into the epidural space; a lead blank that can be optionally used during the implantation procedure to clear a path for the introduction of the leads 106 into the epidural space; a tunneling tool that can be used to create a subcutaneous tunnel from the implantation site of the pulse generator 104 to the implant location of the lead 106; connector plug/port plugs that can be used to seal the port(s) of the pulse generator 104 that are not in use; a cable extension that can be used to connect the leads 106 to the external trial stimulator 102 during intraoperative testing and trial phase; and external adaptors that can be used for connecting other types of leads to external stimulators during in-office evaluation.

The release of the electrical pulses by the electrical contacts 108 of the leads 106 when implanted inside the epidural space causes neuromodulation of the dorsal column, which is responsible for the regulation of pain. The neuromodulation overrides the dysregulation of the spinothalamic tract by suppressing neurons within the dorsal horn of the spinal cord using the electrical pulses that can be distributed in a variety of patterns. The neuromodulation is able to mitigate chronic intractable pain of the trunk and/or limbs. Depending on where the electrical contacts 108 of the leads 106 are positioned in the epidural space, the neuromodulation can mitigate unilateral or bilateral pain associated with failed back surgery syndrome; complex regional pain syndrome (CRPS) Types I and II; intractable lower back pain and leg pain with or without prior back surgery; diabetic peripheral neuropathy of the lower extremities; radicular pain syndrome; radiculopathies resulting in pain secondary to failed back syndrome or herniated disc; epidural fibrosis; degenerative disc disease (herniated disc pain refractory to conservative and surgical interventions); arachnoiditis; multiple back surgeries.

Additionally, the neuromodulation provided by the system 100 can also effectively activate the sympathetic efferent nerves to cause bronchodilation through beta-adrenergic receptors which are abundant on airway smooth muscles. The neuromodulation also inhibits the parasympathetic nerve fibers to prevent bronchoconstriction through both afferent signaling and blocking the efferent fibers. Further, it is contemplated that the neuromodulation targets the visceral afferent nerves in the intermediate zone of the spinal cord which is the region of gray matter that lies between the posterior (or dorsal) and anterior (or ventral) horns of the spinal cord, and includes the rexed lamina X. When the electrical contacts 108 are implanted inside the targeted area of the epidural space between the C7 and T6 vertebrae (or in some instances between the T1 and T4 vertebrae), the neuromodulation provided by the system 100 activates the sympathetic pulmonary axis which inhibits the parasympathetic innervation of the bronchial smooth muscles, preventing constriction of the bronchial smooth muscles. Also, the neuromodulation directly activates the sympathetic nerve fibers to activate beta-adrenergic receptors further supporting relaxation of bronchial smooth muscles. Thus, through stimulation of the dorsal spinal cord region between C7 and T6 vertebrae (or in some instances between the T1 and T4 vertebrae), the system 100 can maintain bronchodilation to mitigate symptoms associated with moderate to severe asthma, COPD, and other chronic pulmonary diseases.

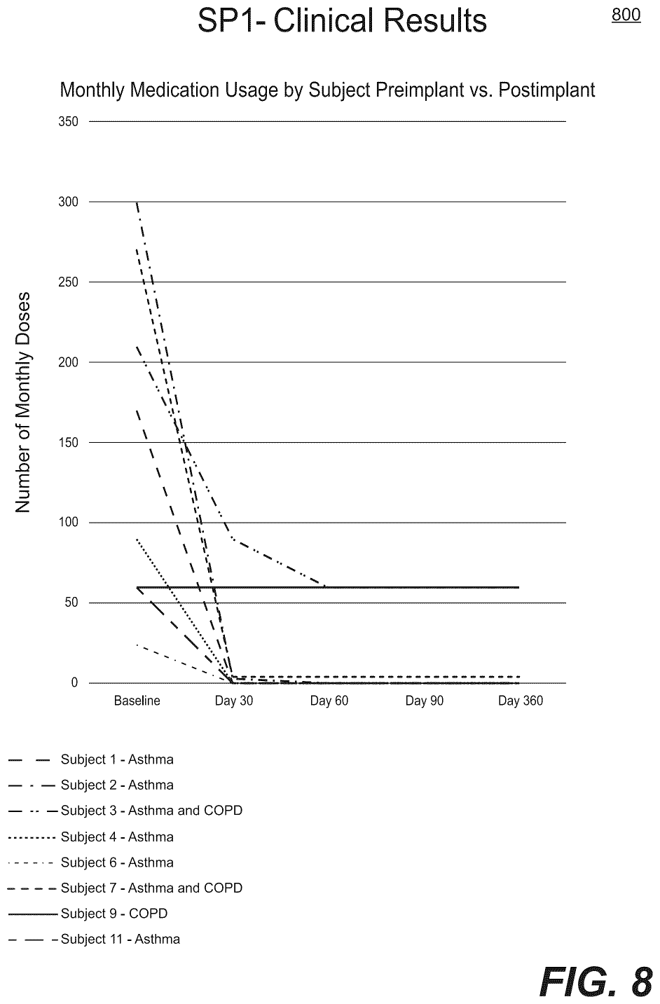

Vagal parasympathetic innervation of airways play a pivotal role in the onset of symptoms associated with inflammatory airway diseases like asthma and COPD. Different mediators released during inflammation, such as interleukin (IL)-4, interleukin (IL)-5, and interleukin (IL)-13, can activate and sensitize both peripheral and central parasympathetic neurons resulting in bronchoconstriction and increased mucus production. Suppression of inflammation using steroids or antibodies targeting cytokines, IgE, and specific cells, is typically not sufficient as neuronal sensitization may be long-lasting and becomes independent leading to limited efficacy. Clinical data, as described further below with reference to FIGS. 8-12, suggests that neuromodulation of specific sympathetic neurons by the system 100 represents a more effective pathway for the treatment of asthma by causing both bronchodilation and blocking parasympathetic activation and sensitization by immune mediators.

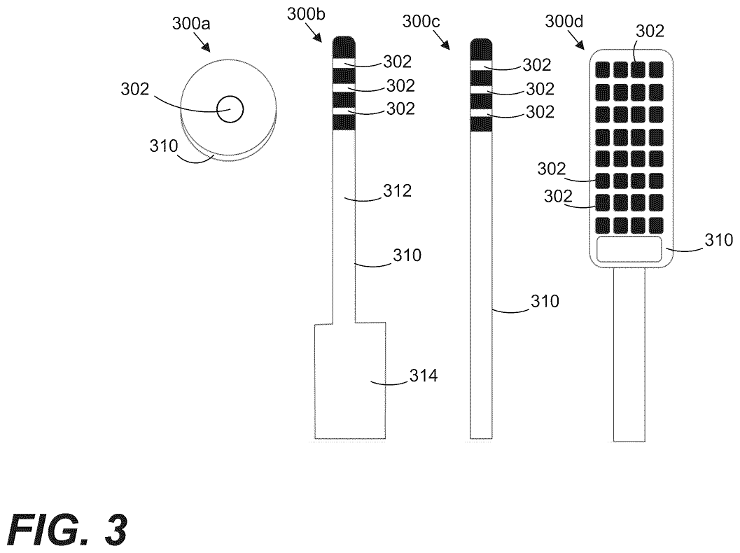

FIG. 3 illustrates examples of additional types of stimulators 300a-300d that can be used to mitigate symptoms caused by chronic respiratory conditions such as moderate to severe asthma and COPD. The stimulators 300a-300d can be placed percutaneously, minimally invasively, or via open surgery. The stimulators 300a-300d release electrical pulses to stimulate neural tissue including sympathetic ganglia proximate to and/or inside of the targeted area A of the epidural space (see FIG. 2). The stimulation provided by the electrical pulses can mitigate symptoms such as wheezing, shortness of breath, chest tightness, and coughing, which can be caused by respiratory conditions such as asthma, COPD, and other chronic pulmonary diseases.

The stimulators 300a-300d do not physically connect or disconnect to a separate pulse generator such as the pulse generator 104 shown in FIG. 1. Instead, the stimulators 300a-300d can each include an internal power source that generates the electrical pulses for release in the targeted area A of the epidural space. The internal power source housed on the stimulators 300a-300d can include one or more rechargeable batteries that can be recharged through wireless energy transfer from an external power source like the charger 110 shown in FIG. 1.

Alternatively, the stimulators 300a-300d can wirelessly receive the electrical pulses from the external power source for releasing the electrical pulses in the targeted area A of the epidural space. Examples of wireless energy transfer for recharging the internal power source of the stimulators 300a-300d and/or for transferring the electrical pulses generated by the external power source to the stimulators 300a-300d can include inductance, midfield radiofrequency (RF), far-field RF, high frequency electromagnetic coupling, ultrasound energy transfer (UET), hypersound, and other wireless power communication techniques.

The stimulators 300a-300d each include one or more electrical contacts 302 mounted on a body 310. The one or more electrical contacts 302 generate an electric field to stimulate the dorsal spinal cord region. The electrical contacts 302 may be cylindrical, semi-cylindrical, spherical, semi-spherical, or planar. In some examples, the electrical contacts 302 can be positioned along a length between a distal end and a proximal end of the body 310 that correlates with a length of the targeted area A of the epidural space between C7 and T6 vertebrae (and in some instances, more specifically, between the T1 and T4 vertebrae).

The stimulator 300a includes a body 310 that is disc shaped. In this example, the stimulator 300a is implantable via open surgery outside of the epidural space, but proximate to the targeted area A of the epidural space. In this manner, the electrical pulses released by the stimulator 300a can stimulate sympathetic ganglia including spinal nerve roots and/or dorsal root ganglion (DRG) next to the targeted area A of the epidural space, but not from inside the epidural space. The stimulator 300a can house an internal power source for generating the electrical pulses. The internal power source can include a rechargeable battery.

The stimulator 300b includes a body 310 that has tubular portion 312 and a housing portion 314 for housing components such as an internal power source. The tubular portion 312 is implantable inside the epidural space, while the housing portion 314 can remain outside of the epidural space. The body 310 of the stimulator 300b including the housing portion 314 can fold for deployment through a catheter such as an introducer sheath or epidural needle, and can then unfold once the tubular portion 312 of the body 310 is implanted inside the epidural space.

The stimulator 300c includes a body 310 that is tubular along its entire length. The stimulator 300c is implantable percutaneously through an introducer sheath or epidural needle. In some examples, the stimulator 300c is a temporary percutaneous implant, such as for conducting a spinal cord stimulation trial. Alternatively, the stimulator 300c can be a permanent percutaneous implant, such as after completion of a successful spinal cord stimulation trial. The stimulator 300c can house an internal power source for generating the electrical pulses, or can receive the electrical pulses from an external power source.

The stimulator 300d is an example of a paddle lead. The stimulator 300d can be permanently implanted either via a minimally invasive procedure or via open surgery. Like in the examples described above, the stimulator 300c can house an internal power source for generating the electrical pulses. Alternatively, the stimulator 300c can receive the electrical pulses from an external power source to provide the neuromodulation in the targeted area A.

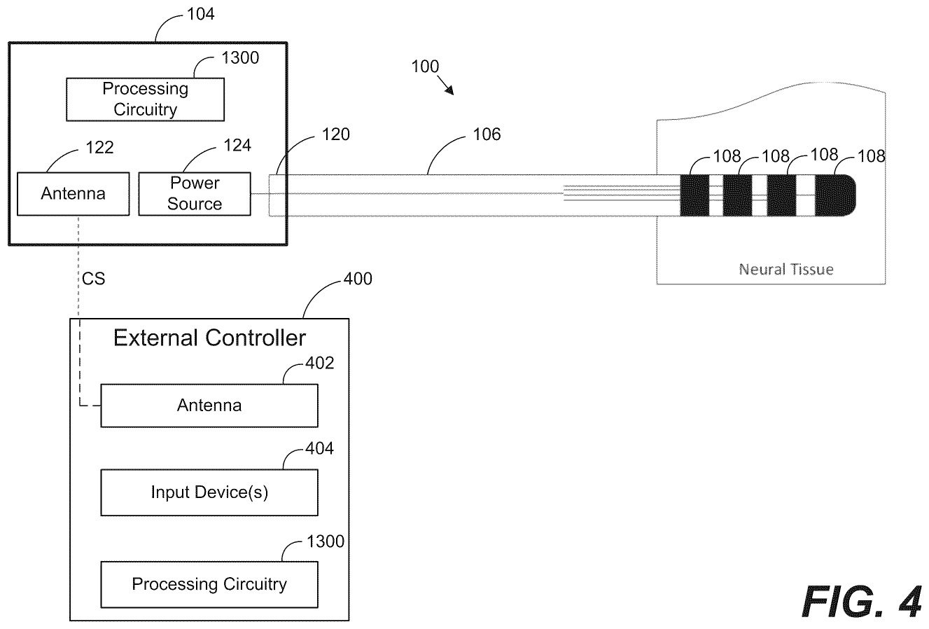

FIG. 4 schematically illustrates an example of the system 100 having a distal end of the lead 106 implanted in proximity to neural tissue in the targeted area A of the epidural space, and a proximal end of the lead 106 plugged into the pulse generator 104. An external controller 400 wirelessly communicates with the pulse generator 104 to program the pulse generator 104 to generate the electrical pulses to have optimal parameters to prevent and/or alleviate symptoms such as wheezing, shortness of breath, chest tightness, and coughing caused by asthma, COPD, and other chronic pulmonary diseases. In some examples, the electrical pulse generated by the pulse generator 104 are programmed such that they are sub-perception such that there is no sensible artifact or side-effect felt by the patient.

As shown in FIG. 4, the system 100 includes the pulse generator 104, the lead 106 having electrical contacts 108 implanted in the epidural space, and the external controller 400. In this example, the lead 106 is plugged into a receptacle 120 of the pulse generator 104 such that the lead 106 is mechanically connected to the pulse generator 104. As described above, the pulse generator 104 generates the electrical pulse for transmission through the lead 106. The electrical pulses are released by the electrical contacts 108 to neuromodulate the neural tissue in the targeted area A of the epidural space which is located between the C7 and T6 vertebrae (and in some instances, more specifically, between the T1 and T4 vertebrae).

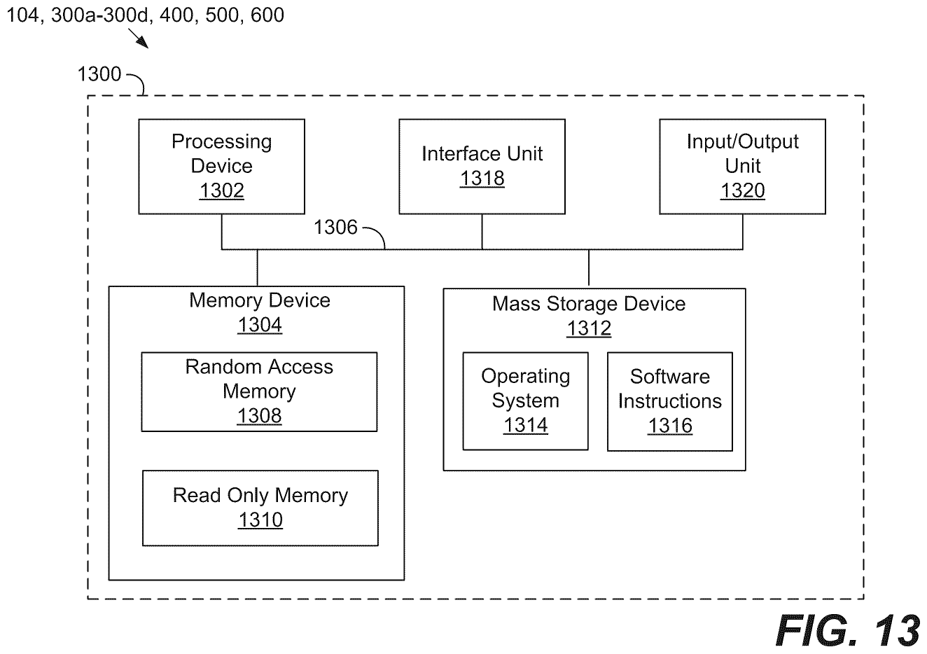

The pulse generator 104 includes an antenna 122, an internal power source 124, and a processing circuitry 1300 that can include non-transitory computer readable storage media for storing parameters such as an amplitude, a frequency, and a pulse width that are optimal for mitigating symptoms associated with chronic pulmonary diseases. An example of the processing circuitry 1300 is shown in FIG. 13, which is described in more detail further below.

In some examples, the antenna 122 is a passive antenna such that it only receives transmissions from the external controller 400. In alternative examples, the antenna 122 is an active antenna allowing two-way communications between the pulse generator 104 and the external controller 400 such as to share data, control signals, and other transmissions.

The external controller 400 includes a processing circuitry 1300 operatively connected to an antenna 402 and one or more input devices 404. The antenna 402 wirelessly communicates with the antenna 122 of the pulse generator 104 to program the pulse generator 104 to generate the electrical pulses to have the predetermined combination of the amplitude, the frequency, and the pulse width to target bronchodilation and/or prevent bronchoconstriction.

The antenna 402 can transmit control signals CS for programming the pulse generator 104 based on the inputs received by the one or more input devices 404. The one or more input devices 404 can include push buttons and/or a touchscreen display such as when the external controller 400 is a tablet computer like the one shown in FIG. 1. The antenna 402 can wirelessly communicate with the antenna 122 of the pulse generator 104 via Bluetooth, Wi-Fi, Zigbee, near-field communication (NFC), or other wireless communications protocols.

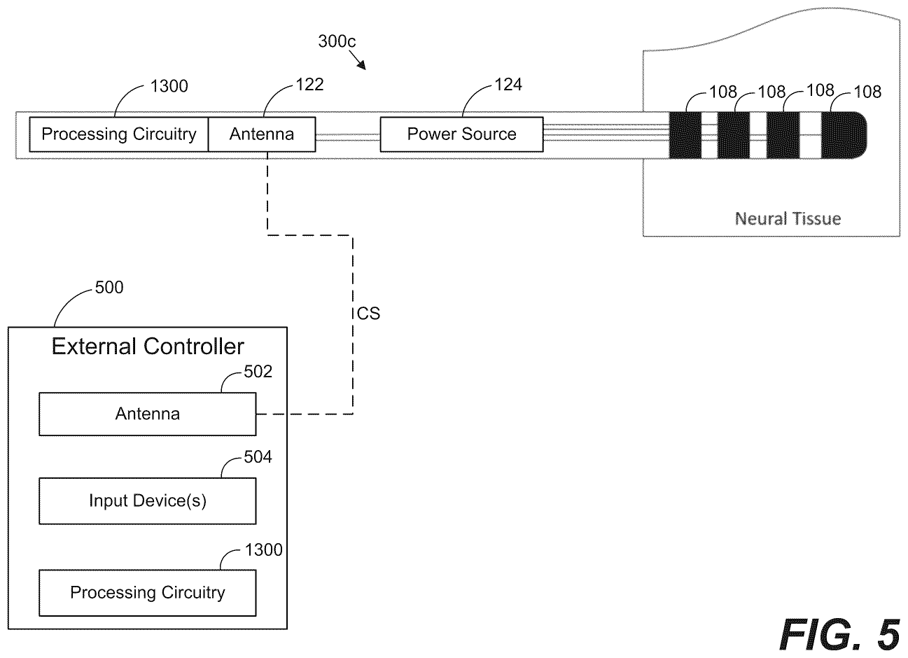

FIG. 5 schematically illustrates another example of the stimulator 300c having a distal end implanted in proximity to neural tissue in the targeted area A of the epidural space. In this example, the stimulator 300c is a leadless stimulator because the stimulator 300c does not include leads attached to a pulse generator. An external controller 500 wirelessly communicates with the stimulator 300c to program the stimulator 300c to generate the electrical pulses to have optimal parameters to prevent and/or alleviate symptoms such as wheezing, shortness of breath, chest tightness, and coughing caused by asthma, COPD, and other chronic pulmonary diseases.

In FIG. 5, the stimulator 300c has an internal power source 124 and electrical contacts 108 implanted in the neural tissue. The internal power source 124 generates the electrical pulses. The electrical contacts 108 release the electrical pulses to neuromodulate the neural tissue in the targeted area A of the epidural space located between the C7 and T6 vertebrae (and in some instances, more specifically, between the T1 and T4 vertebrae).

The stimulator 300c further includes an antenna 122 and a processing circuitry 1300 that can include non-transitory computer readable storage media that stores neuromodulation parameters such as the predetermined combination of the amplitude, the frequency, and the pulse width that are optimal for mitigating symptoms associated with chronic pulmonary diseases.

In some examples, the antenna 122 is a passive antenna such that it only receives transmissions from the external controller 500. In alternative examples, the antenna 122 is an active antenna allowing two-way communications between the stimulator 300c and the external controller 500 such as to share data, control signals, and other transmissions.

The external controller 500 includes a processing circuitry 1300 operatively connected to an antenna 502 and one or more input devices 504. The antenna 502 wirelessly communicates with the antenna 122 of the stimulator 300c to program the stimulator 300c to generate the electrical pulses to have the predetermined combination of the amplitude, the frequency, and the pulse width to target bronchodilation and/or prevent bronchoconstriction.

The antenna 502 can transmit control signals CS for programming the stimulator 300c based on the inputs received by the one or more input devices 504. The one or more input devices 504 can include push buttons and/or a touchscreen display such as when the external controller 500 is a tablet computer like the one shown in FIG. 1. The antenna 502 can wirelessly communicate with the antenna 122 of the stimulator 300c via Bluetooth, Wi-Fi, Zigbee, near-field communication (NFC), or other wireless communications protocols.

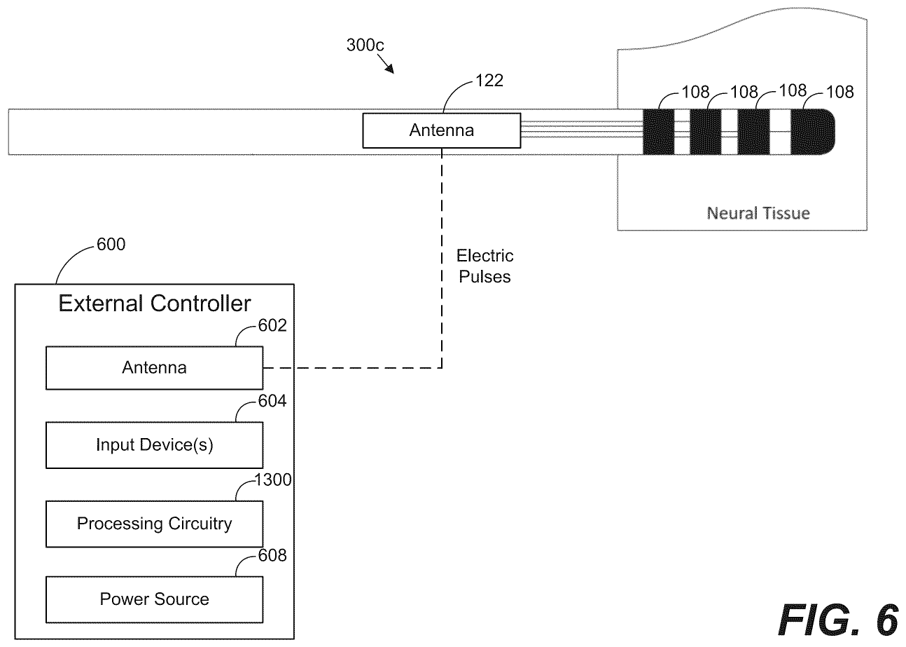

FIG. 6 schematically illustrates another example of the stimulator 300c having a distal end implanted in proximity to neural tissue in the targeted area A of the epidural space. In this example, the stimulator 300c is a leadless stimulator because the stimulator 300c does not include leads attached to a pulse generator. An external controller 600 wirelessly communicates with the stimulator 300c to control the stimulator 300c to release the electrical pulses having the neuromodulation parameters that are optimal to prevent and/or alleviate symptoms such as wheezing, shortness of breath, chest tightness, and coughing caused by asthma, COPD, and other chronic pulmonary diseases.

In FIG. 6, the stimulator 300c includes electrical contacts 108 implanted in proximity to the epidural neural tissue. The stimulator 300c does not include an internal power source. Instead, an external power source 608 is included on the external controller 600. The external power source 608 can include one or more batteries such as rechargeable or disposable batteries. The external power source 608 generates the electrical pulses which can be transferred to the stimulator 300c for release by the electrical contacts 108 to neuromodulate the neural tissue in the targeted area A of the epidural space located between the C7 and T6 vertebrae (and in some instances, more specifically, between the T1 and T4 vertebrae).

In some examples, the stimulator 300c receives subcutaneous energy from an external device such as a transcutaneous electrical nerve stimulation (TENS) unit or similar type of device. The energy is transmitted through the skin toward a proximal end of the stimulator 300c, and then the conducted energy is transferred to the electrical contacts 108 at a distal end or tip of the stimulator 300c for stimulating a neural target of the spinal nerves and/or DRG.

The external controller 600 includes an antenna 602 that can transfer the electrical pulses generated by the external power source 608 to the antenna 122 on the stimulator 300c for release by the electrical contacts implanted in proximity to the targeted area A of the epidural space. The electrical pulses can be transferred from the antenna 602 to the antenna 122 using the wireless energy transfer techniques described above such as inductance, midfield radiofrequency (RF), far-field RF, high frequency electromagnetic coupling, ultrasound energy transfer (UET), hypersound, and other wireless energy transfer techniques. In this example, the antenna 122 is a passive antenna such that it only receives transmissions from the external controller 600.

The external controller 600 includes a processing circuitry 1300 operatively connected to an antenna 602, one or more input devices 604, and the external power source 608. The processing circuitry 1300 controls the external power source 608 to generate the electrical pulses to have the predetermined combination of the amplitude, the frequency, and the pulse width to target bronchodilation and/or prevent bronchoconstriction based on the neuromodulation parameters selected or entered by a user of the external controller 600.

Table 1 provides value ranges for the neuromodulation parameters that are used to target bronchodilation and/or prevent bronchoconstriction by the electrical pulses released by the electrical contacts 108 positioned proximate to and/or inside of the targeted area A of the epidural space between the C7 and T6 vertebrae (and in some instances, more specifically, between the T1 and T4 vertebrae). The neuromodulation parameters include an amplitude that defines a strength of the electrical pulses delivered to the neural tissue in the targeted area A, a pulse width that defines a duration of the electrical pulses, and a frequency that defines how often the electrical pulses are delivered to the neural tissue in the targeted area A.

TABLE 1

| Parameter | Value Range |
|---|---|
| Amplitude (mAmp) | 0.5-2.5 |
| Frequency (Hz) | 350-500 |
| Pulse Width (μs) | 240-350 |

The external controllers 400-600 can be used to program the pulse generator 104 of the system 100 and the stimulators 300a-300d to generate the electrical pulses having the amplitude, frequency, and pulse width values defined in Table 1. The amplitude, frequency, and pulse width values defined in Table 1 can be stored on non-transitory computer readable storage media housed on the pulse generator 104 of the system 100. Additionally, the amplitude, frequency, and the pulse width values defined in Table 1 can be stored on non-transitory computer readable storage media housed on the stimulators 300a-300d. The pulse generator 104 and the internal power sources 124 can then generate the electrical pulses having the amplitude, frequency, and pulse width values defined in Table 1 for delivery to the neural tissue including the sympathetic ganglia in the targeted area A of the epidural space.

In some examples, prior to programming the pulse generator 104 of the system 100 and the stimulators 300a-300d to generate the electrical pulses having the amplitude, frequency, and pulse width values defined in Table 1, a test procedure can be performed to confirm stimulation of the chest area. The test procedure can be performed when implanting the external trial stimulator 102 such as for a trial stimulation. The test procedure can include controlling the external trial stimulator 102 to emit electrical pulses having a low frequency of about 30 Hz to about 50 Hz, a pulse width of about 150 mAmp to about 300 mAmp, and then gradually increasing the amplitude until a perception threshold is reached such that the patient feels a tingling or vibration sensation (similar to that of paresthesia) in the chest area. Once the patient confirms the tingling or vibration sensation in the chest area, the external trial stimulator 102 can be programmed to have the parameters defined in Table 1 such that the spinal cord stimulation directed to the chest area of the patient is sub-perception.

In examples where the stimulators 300a-300d do not include an internal power source, the amplitude, frequency, and pulse width values defined in Table 1 can be stored on non-transitory computer readable storage media housed on the external controller 600. The processing circuitry 1300 of the external controller 600 controls the external power source 608 to generate the electrical pulse having the amplitude, frequency, and pulse width values defined in Table 1. The electrical pulses are transferred from the external controller 600 to the stimulators 300a-300d for delivery by the electrical contacts 108 to the neural tissue including the sympathetic ganglia in the targeted area A of the epidural space.

The amplitude, the frequency, and the pulse width values for the neuromodulation to mitigate symptoms associated with chronic respiratory conditions are unexpected because these value ranges can be different from the value ranges that are typically used for treating chronic pain. For example, when mitigating symptoms associated with chronic respiratory conditions, the system 100 and the stimulators 300a-300d operate at comparatively low amplitudes, frequencies, and pulse widths than those typically used for chronic pain management. The system 100 and the stimulators 300a-300d are generally approved to operate within large value ranges such as an amplitude ranging from 0 to 25.5 (mAmp), a frequency ranging from 2-1,200 (Hz), and a pulse width range from 20 to 1,000 μs, whereas, as noted in Table 1, the neuromodulation parameters for treating chronic respiratory conditions operate at comparatively low amplitudes, frequencies, and pulse widths than those generally approved for the system 100.

Further, the amplitude, the frequency, and the pulse width values for the neuromodulation to mitigate symptoms associated with chronic respiratory conditions are unexpected because, in at least some example implementations, the neuromodulation does not cycle on and off like what is typically done to mitigate chronic pain. Instead, the neuromodulation for mitigating symptoms associated with chronic respiratory conditions can remain on at all times to prevent bronchoconstriction. Given the foregoing, the amplitude, the frequency, and the pulse width value ranges identified in Table 1 are not easily derivable by trial and error because when cycled on and off like what is typically done to mitigate chronic pain, the neuromodulation by the system 100 may not be as effective in mitigating symptoms associated with chronic respiratory conditions.

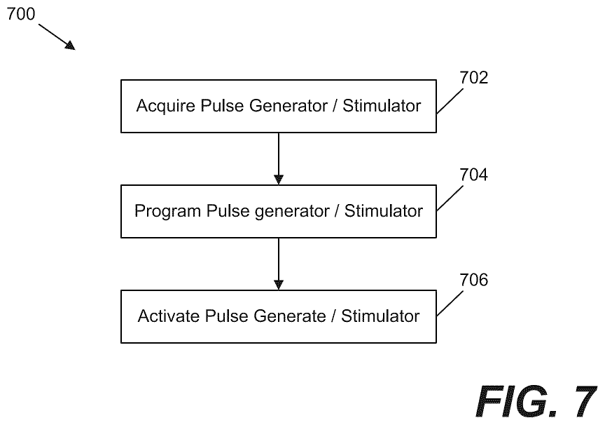

FIG. 7 schematically illustrates an example of a method 700 of treating respiratory conditions using spinal cord stimulation. The method 700 can include using the system 100 of FIG. 1 or the stimulators 300a-300d of FIG. 3. Advantageously, the method 700 can prevent onset of bronchospasms or relieve asthma attacks without drug intervention.

The method 700 includes an operation 702 of acquiring the pulse generator 104 which, as described above, is configured for spinal cord stimulation. Alternatively, operation 702 can include acquiring one of the stimulators 300a-300d which are also configured for spinal cord stimulation. As discussed above, the pulse generator 104 and the stimulators 300a-300d are generally configured to provide spinal cord stimulation to mitigate chronic pain in patients.

The method 700 includes an operation 704 of programming the pulse generator 104 of the system 100 shown in FIG. 1 to generate electrical pulses to provide a neuromodulation that prevents bronchoconstriction. Alternatively, operation 704 can include programming the stimulators 300a-300d shown in FIG. 3 to generate the electrical pulses to provide the neuromodulation that prevents bronchoconstriction.

In operation 704, the pulse generator 104 and the stimulators 300a-300d are programmed to generate the electrical pulses for providing neuromodulation targeting the thoracic region of the spinal cord. Further, the neuromodulation is programmed to stimulate a dorsal spinal cord region causing bronchodilation and/or prevent constriction of the bronchial smooth muscles. For example, the pulse generator 104 and the stimulators 300a-300d are programmed to generate the electrical pulses for targeting sympathetic ganglia between the C7 and T6 vertebrae (or in some instances between the T1 and T4 vertebrae).

In operation 704, the pulse generator 104 and the stimulators 300a-300d are programmed to provide the neuromodulation having parameters that include an amplitude that ranges from about 0.5 mAmp to about 2.5 mAmp, a predefined frequency that ranges from about 350 Hz to about 500 Hz, and a predefined pulse width that ranges from about 240 μs to about 350 μs. As discussed above, the values of the amplitude, the frequency, and the pulse width can sustain bronchodilation and/or prevent bronchoconstriction. Accordingly, the neuromodulation prevents onset of asthma attacks without drug intervention. Thus, the neuromodulation can mitigate symptoms associated with moderate and severe asthma.

In some examples, the method 700 can include an operation 706 of activating the pulse generator 104 after implantation inside a patient. Alternatively, operation 706 of activating one of the stimulators 300a-300d after implantation inside a patient. Activating the pulse generator 104 and the stimulators 300a-300d causes these devices to provide the neuromodulation in accordance with the programming done in operation 704.

The method 700 as a treatment modality for asthma presents a paradigm shift in the management of this chronic respiratory condition. Currently, the standard of care for asthma primarily relies on the frequent use of inhaled corticosteroids (ICS) and long-acting beta agonists (LABA) inhalers, nebulizers, oral medications, and rescue inhalers. However, this regimen often necessitates multiple daily doses, leading to inconvenience and dependency on steroids with various adverse effects, including but not limited to inflammation, tremors, nervousness, and insomnia. In contrast, the method 700 offers a novel approach by eliminating the need for these standard of care treatments altogether, thereby addressing the limitations associated with conventional asthma treatments.

At least one significant advantage of the method 700 is the potential to induce remission in asthma patients, allowing them to discontinue medication usage entirely. Continuous stimulation provided by the system 100 or stimulators 300a-300d programmed to follow the neurostimulation parameters identified in Table 1 can help to prevent onset of symptoms associated with moderate to severe asthma, which differs from the approach taken by medications that provide relief from asthma symptoms after their onset. By offering a more sustainable and effective alternative to traditional treatments, the method 700 can enhance patient outcomes and also minimize the apprehension often associated with the long-term use of asthma medications, particularly among those concerned about potential side effects.

Moreover, asthma, characterized by its many triggers that present themselves in everyday environments, significantly impacts patients' quality of life and limits their ability to engage in routine activities. The system 100 and stimulators 300a-300d when programmed for the treatment of asthma as disclosed herein can mitigate these challenges by providing patients with greater freedom and flexibility in managing their chronic respiratory condition. With these implantable devices, patients may experience improved symptom control and reduced susceptibility to environmental triggers, allowing them to pursue a more fulfilling and unrestricted lifestyle. By alleviating the need for constant interruption for treatment and the avoidance of asthmatic triggers, the system 100 and stimulators 300a-300d when programmed for the treatment of asthma as disclosed herein empowers asthma patients to reclaim control over their health and enjoy a higher degree of autonomy and peace of mind in their daily lives.

A pilot study was conducted to determine the effects of spinal cord stimulation on subjects who experience both chronic pain and are diagnosed with asthma and/or chronic obstructive pulmonary disease (COPD). A set of eleven subjects were implanted with the system 100 programmed to provide the neuromodulation parameters described above. Among the eleven subjects, eight were diagnosed with chronic pain of the neck or back and moderate-to-severe asthma, two were diagnosed with chronic pain of the neck or back and moderate-to-severe asthma and COPD, and one was diagnosed with both chronic pain of the neck or back and COPD. The study gathered preliminary data concerning the effects of spial cord stimulation on the frequency of asthma medication usage and metrics used to determine patient quality of life.

Of the eleven subjects enrolled, three subjects had been lost to follow-up due to relocation. Four subjects (including the three subjects lost to relocation and one subject whose medication dosage information was not recorded) were not considered in the analyses of asthma medication usage frequency before and after implantation of the system 100. However, nine of the eleven subjects reported all data from their quality of life assessments.

The subjects underwent both trial and permanent implantation procedures. The three day trial period was used to ensure that the subjects would respond to spinal cord stimulation, during which they were evaluated for a reduction in pain of over 50% (measured with a visual analog scale) and a reduction in asthma symptoms (e.g., wheezing) and in asthma medication use. During the trial period, providers used fluoroscopy to determine and document appropriate lead positioning. After the trial period, the implanted leads were extracted.

Each subject who successfully completed the trial period without experiencing any adverse events received a permanent implant of the pulse generator 104. The subjects were intraoperatively tested with a proprietary algorithm to ensure the stimulation was providing relief of both asthma symptoms and pain in the targeted areas. The pulse generators 104 were programmed after the implantation procedure, and once programmed, were turned on from that point forward. The stimulation parameters set on the pulse generators 104 for each subject remained constant throughout the observational period, and each subject's device was set to different parameters depending on what settings the subject responded best to during intraoperative testing. The stimulation parameters were within the value ranges in Table 1.

Asthma and COPD medication dosage data was collected for 180 days following implantation of the pulse generators 104 programmed with the stimulation parameters for treating asthma and COPD. The subjects were not asked to stop using their medications at any point. Nevertheless, the subjects reported rapid improvement in asthma symptoms, which led to largely reduced medication usage over time. The subjects also indicated cessation of wheezing.

FIG. 8 is a chart 800 showing average monthly asthma and COPD medication dosage (Y-axis) over duration of time (X-axis) by the subjects of the pilot study. The data in the chart 800 is based on implantation of the system 100 programmed with the stimulation parameters within the value ranges shown in Table 1. The data in the chart 800 considers subjects who used various medications including rescue inhalers, oral medications, and nebulizers. For the subjects that used inhalers, each puff of an inhaler was considered one dose of the medication.

As shown in FIG. 8, the subjects diagnosed with asthma alone saw more significant decline in the average number of asthma medication doses taken each month. The two subjects with asthma and COPD saw decreases in medication usage, which plateaued after 30 days. The subject diagnosed with COPD alone saw no change in their medication usage. In considering that medication dosage frequency in patients with asthma alone was most significantly affected by the spinal cord stimulation, these patients were then analyzed separately from those with COPD.

The demographics of the asthma-only subject group are listed in Table 2. Of the four subjects, two subjects were implanted with two of the pulse generators 104, where the electrodes for one pulse generator were positioned inside the epidural space between the T1-T6 vertebrae for the treatment of asthma, while the electrodes of the other pulse generator were positioned inside the epidural space between the T7-T9 vertebrae for the treatment of chronic pain.

TABLE 2

| Metric | Value |
|---|---|
| Sample Size | N = 4 |
| Gender | 1 male/3 female |
| Average Age | 54 (range: 32-72) |
| Moderate/Severe Asthma | 50% moderate/50% severe |
| Quantity of Smokers | 2 |
| Quantity of Subjects with Multiple | 2 |
| Pulse Generators Implanted (1 for | |
| Pain, 1 for Asthma) | |

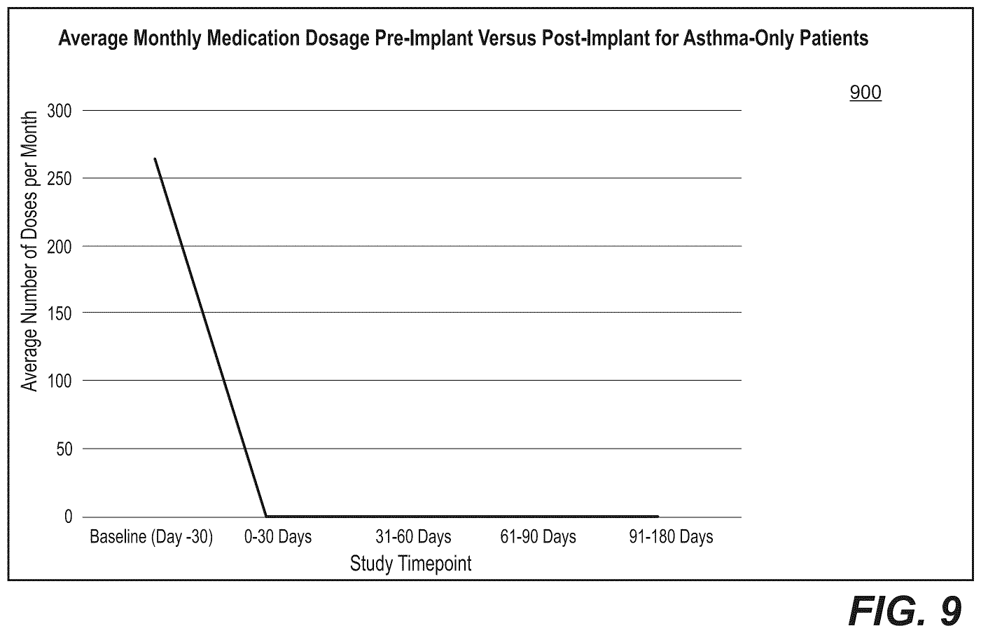

FIG. 9 illustrates a chart 900 showing average number of medication doses taken per month (Y-axis) over time (X-axis) for the asthma-only subject group identified in Table 2. Overall, the mean monthly use of rescue inhalers decreased over an initial 30-day period following implantation of the system 100 with the stimulation parameters within the value ranges shown in Table 1 from 266 doses per month to 0.75 doses per month. After 30 days, two of the four subjects stopped using asthma medication entirely. One subject who continued to use a rescue inhaler experienced mild symptoms every three days on average. This subject's symptoms subsided completely after 180 days, allowing them to cease medication usage. Another subject contracted COVID-19 during the study, and this necessitated a temporary rescue inhaler use for three days. Following recovery from COVID-19, this subject stopped using the rescue inhaler again. Apart from these two cases where subjects utilized rescue inhalers, subjects eliminated the use of all other asthma medications they were previously taking before implantation of the system 100 programmed with the stimulation parameters within the value ranges shown in Table 1. Overall, asthma medication usage decreased by 99.7% amongst the four subjects observed with only chronic pain and moderate-to-severe asthma.

Of the eleven subjects enrolled in the study, nine subjects, diagnosed with either asthma alone, asthma and COPD, or COPD alone, provided disease assessment scores, including an asthma control test (ACT), asthma control questionnaire (ACQ), and asthma quality of life questionnaire (AQLQ), before and after implantation of the system 100.

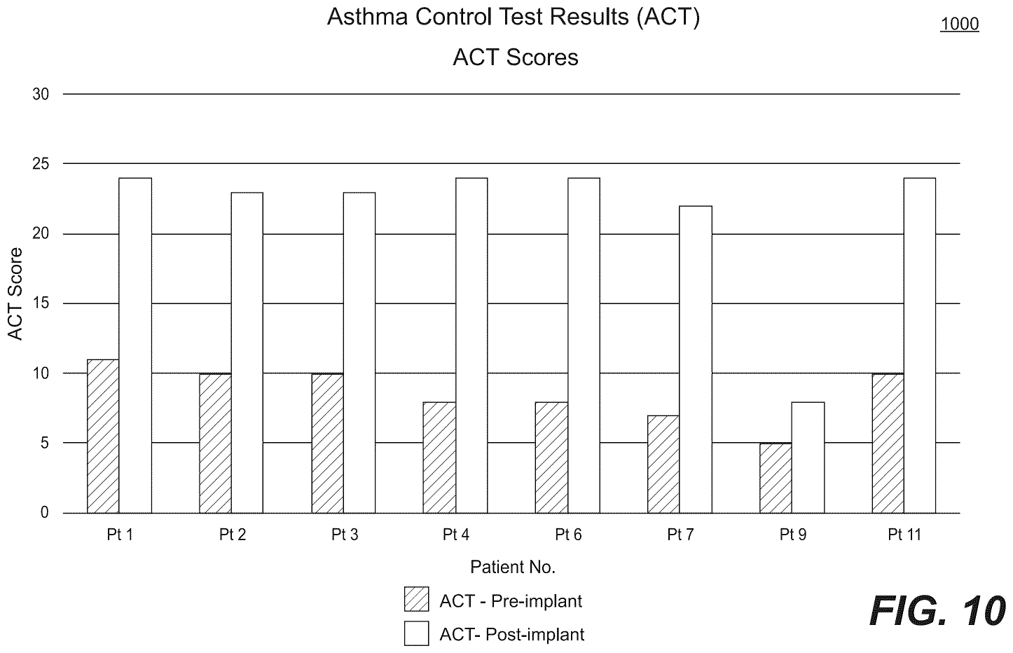

FIG. 10 illustrates a chart 1000 showing ACT scores for the subjects of the pilot study. The ACT is a clinically validated questionnaire used to gauge asthma control based on symptoms, medication use, and daily impact as recalled by the subjects over the past four weeks. Scores can range from 5 to 25, where higher scores indicate that the subjects have better control over their condition. A score of 19 or higher indicates controlled asthma, while a score of less than 19 indicates uncontrolled asthma. As is shown in FIG. 10, all subjects who reported their ACT scores before and after implantation of the system 100 exhibited increased ACT scores. Following implantation, eight out of nine subjects received scores over 19, demonstrating their asthma to be controlled. The only subject who did not receive a score of 19 or higher following implantation (i.e., subject #9), was the only patient diagnosed with COPD alone.

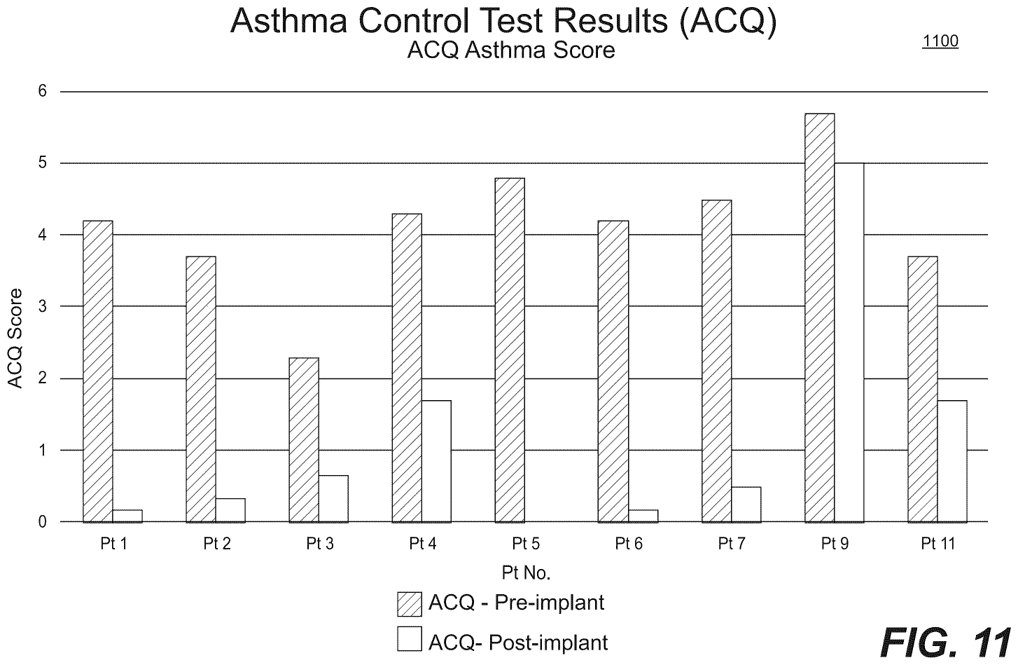

FIG. 11 illustrates a chart 1100 showing ACQ scores for the subjects of the pilot study. The ACQ is similar to the ACT in that it is another validated metric that evaluates asthma control through symptoms and medication use, but the ACQ differs in that it also measures lung function and considers information recalled by the subjects over a previous week. The ACQ provides a score indicating the level of control ranging from 0 to 6, where lower scores indicate better control. As is shown in FIG. 11, all patients saw decreased ACQ scores following implantation of the system 100. Once again, the condition of subject #9, the patient with only COPD, was not impacted as significantly as that of the other patients who received the system 100 programmed with the stimulation parameters within the value ranges shown in Table 1.

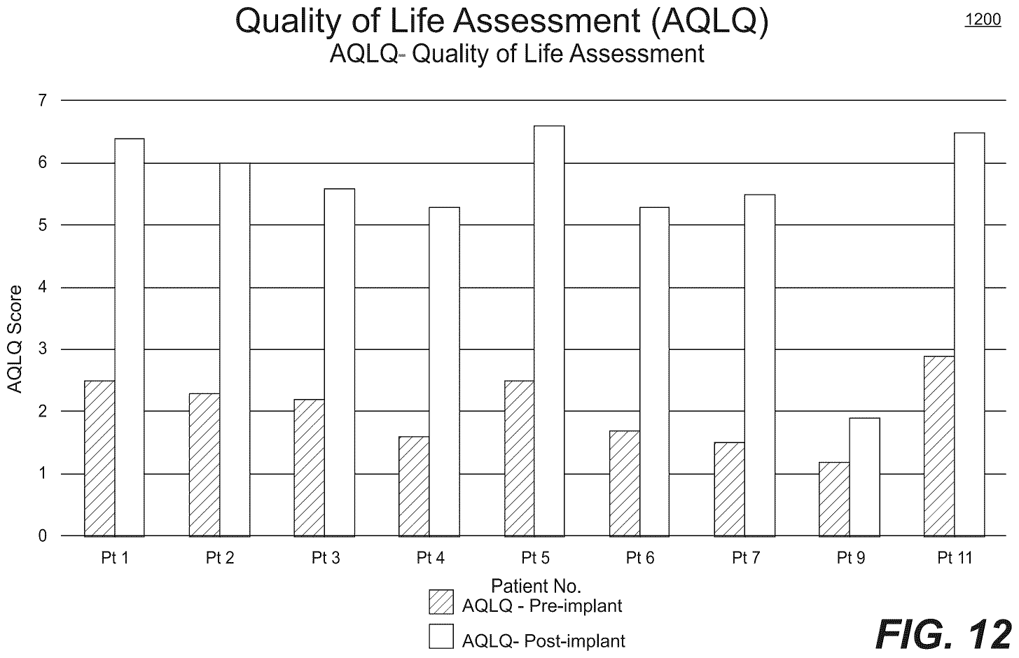

FIG. 12 illustrates a chart 1200 showing AQLQ scores for the subjects of the pilot study. The AQLQ (along with the standardized version of the assessment, AQLQ(S)) is a validated metric that measures asthma's impact on quality of life across physical, emotional, social, and occupational aspects, typically through subject-rated questions. These four domains make up a 32-question test, where subjects are asked to consider their responses over the last two weeks. Scores for the AQLQ and AQLQ(S) range on a scale of 1-7, where higher scores tend to attest to a higher quality of life. As shown in FIG. 12, all subjects experienced improvements in their quality of life and ability to control their asthma following the implantation of the system 100. Through these results, it was demonstrated that all subjects saw some benefit to the implantation of the system 100 programmed with the stimulation parameters within the value ranges shown in Table 1. Subject #9, once again, saw the least improvement in their quality of life. These findings suggest that patients with moderate-to-severe asthma are more likely to respond and see significant changes to their condition than patients without asthma.

The pilot study provides preliminary evidence that use of the system 100 as an electrical bronchodilator can provide improved quality of life and control over chronic respiratory conditions, without the occurrence of any adverse events, for subjects with moderate-to-severe asthma or moderate-to-severe asthma combined with COPD. While the subject with only COPD still benefitted from the implantation of the system 100, the results of the pilot study suggest that patients with moderate-to-severe asthma responded the best overall to the spinal cord stimulation provided by the system 100, and are an ideal target group for this therapy.

FIG. 13 schematically illustrates an example of the processing circuitry 1300 that can be used to implement various aspects described herein including functions performed on the pulse generator 104 of the system 100, the stimulators 300a-300d, and the external controllers 400-600. The processing circuitry 1300 includes a processing device 1302 and a memory device 1304. Examples of the processing device 1302 can include a processor, a processing unit, a central processing unit (CPU), a microprocessor, a digital signal processor, a field-programmable gate array, and other types of processing digital circuits.

The memory device 1304 can include a random-access memory (RAM) 1308 and a read-only memory (ROM) 1310. Basic input and output logic having routines to transfer data between elements in the processing circuitry 1300 can be stored in the ROM 1310.

In some examples, the processing circuitry 1300 can include a mass storage device 1312. The mass storage device 1312 is connected to the processing device 1302 through the system bus 1306. The memory device 1304 and the mass storage device 1312 are examples of computer-readable data storage media that provides non-volatile, non-transitory storage for the processing circuitry 1300. The memory device 1304 and the mass storage device 1312 can store an operating system 1314, software instructions 1316, and/or data for execution by the processing device 1302.

The computer-readable data storage media can be any available non-transitory, physical device or article of manufacture from which the processing circuitry 1300 can read data and/or instructions. Computer-readable data storage media can include removable and non-removable media implemented in any method or technology for storage of information such as computer-readable software instructions, data structures, program modules or other data. Types of computer-readable data storage media include, but are not limited to, RAM, ROM, EPROM, EEPROM, flash memory or other solid-state memory technology, or any other medium which can be used to store information, and which can be accessed by the processing device 1302.

The processing circuitry 1300 can operate in a networked environment using logical connections to other devices. For example, the processing circuitry 1300 can connect to another device through an interface unit 1318. The interface unit 1318 can connect to additional systems and devices through wired connections such as Ethernet, or through Bluetooth, Wi-Fi, Zigbee, and other wireless technologies. The processing circuitry 1300 can also include an input unit 1320 for receiving and processing inputs from input devices such as the external controller 400.

The mass storage device 1312 and the RAM 1308 can store software instructions and data. The software instructions can include an operating system 1314 suitable for controlling the processing circuitry 1300. The mass storage device 1312 and/or the RAM 1308 can also store the software instructions 1316, which when executed by the processing device 1302, cause the processing device 1302 to provide the functionalities disclosed herein.

The various embodiments described above are provided by way of illustration only and should not be construed to be limiting in any way. Various modifications can be made to the embodiments described above without departing from the true spirit and scope of the disclosure.

What is claimed is:

1. A device for treating respiratory conditions, the device comprising:
a processing circuitry having non-transitory computer readable storage media storing instructions which, when executed by the processing circuitry, cause the processing circuitry to:
generate electrical pulses directed to a region of the spinal cord column, the electrical pulses providing a neuromodulation that prevents bronchoconstriction, the neuromodulation having an amplitude ranging from about 0.5 mAmp to about 2.5 mAmp, a frequency ranging from about 350 Hz to about 500 Hz, and a pulse width ranging from about 240 μs to about 350 μs.

2. The device of claim 1, wherein the neuromodulation stimulates a dorsal spinal cord region causing bronchodilation or preventing bronchoconstriction.

3. The device of claim 1, wherein the neuromodulation targets parasympathetic and sympathetic ganglia between the T1 and T4 vertebrae.

4. The device of claim 1, wherein the neuromodulation mitigates symptoms associated with moderate and severe asthma.

5. The device of claim 1, wherein the neuromodulation prevents onset of asthma attacks without drug intervention.

6. The device of claim 1, further comprising:
one or more leads configured to be implanted in an epidural space of the spinal canal, each of the one or more leads including electrical contacts for releasing the electrical pulses inside the epidural space between the T1 and T4 vertebrae.

7. The device of claim 6, wherein the one or leads are temporary percutaneous leads configured to be removed from the epidural space after completion of a spinal cord stimulation trial.

8. The device of claim 6, wherein the one or more leads are permanent leads.

9. The device of claim 6, further comprising:
a pulse generator connected to the one or more leads, and pulse generator housing the processing circuitry having the non-transitory computer readable storage media.

10. A method of treating respiratory conditions, the method comprising:
acquiring a pulse generator configured for neuromodulation; and
programming the pulse generator to generate electrical pulses directed to a region of the spinal cord, the electrical pulses providing the neuromodulation to prevent bronchoconstriction, the neuromodulation having an amplitude ranging from about 0.5 mAmp to about 2.5 mAmp, a frequency ranging from about 350 Hz to about 500 Hz, and a pulse width ranging from about 240 μs to about 350 μs.

11. The method of claim 10, further comprising:
activating the pulse generator after implantation inside a patient.

12. The method of claim 10, further comprising:
providing the neuromodulation to stimulate a dorsal spinal cord region causing bronchodilation or preventing bronchoconstriction.

13. The method of claim 10, further comprising:
providing the neuromodulation to target sympathetic ganglia between the T1 and T4 vertebrae.

14. The method of claim 10, further comprising:
providing the neuromodulation to mitigate symptoms associated with moderate and severe asthma, and chronic obstructive pulmonary disease (COPD).

15. The method of claim 10, further comprising:
providing the neuromodulation prevents onset of asthma attacks without drug intervention.
